(19) United States
(12) Patent Application Publication
(10) Pub. No.: US 2002/0128228 A1
(43) Pub. Date: Sep. 12, 2002

(54) Compositions and methods for the treatment of cancer

(76) Inventor: Wen-Jen Hwu, New York, NY (US)

Correspondence Address: PENNIE AND EDMONDS, 1155 AVENUE OF THE AMERICAS, NEW YORK, NY, 100362711

(21) Appl. No.: 10/001,281

(22) Filed: Nov. 30, 2001

Related U.S. Application Data
(60) Provisional application No. 60/250,130, filed on Dec. 1, 2000.

(51) Int. Cl. A61K 31/724; A61K 31/454

(52) U.S. Cl. 514/58; 514/183; 514/323

(57) ABSTRACT

This invention relates to compositions comprising temozolomide and thalidomide which can be used in the treatment or prevention of cancer, in particular malignant melanoma, cancer of the skin, subcutaneous tissue, lymph nodes, brain, lung, liver, bone, intestine, colon, heart, pancreas, adrenals, kidney, prostate, breast, colorectal, or a combination thereof. A particular composition comprises temozolomide, or a pharmaceutically acceptable salt, solvate, or clathrate thereof, and thalidomide, or a pharmaceutically acceptable salt, solvate, or clathrate thereof. The invention also relates to methods of treating or preventing cancer, in particular malignant melanoma, cancer of the skin, subcutaneous tissue, lymph nodes, brain, lung, liver, bone, intestine, colon, heart, pancreas, adrenals, kidney, prostate, breast, colorectal, or a combination thereof, which comprise the administration of temozolomide and thalidomide and another anti-cancer drug to a patient in need of such treatment or prevention. The invention further relates to methods of reducing or avoiding adverse side effects associated with the administration of cancer chemotherapy or radiation therapy which comprise the administration of temozolomide and thalidomide to a patient in need of such reduction or avoidance.

This application claims priority to U.S. Provisional Application Serial No. 60/250,130 filed Dec. 1, 2000 which is hereby incorporated by reference.

### 1. FIELD OF THE INVENTION

This invention relates to methods of treating primary and metastatic cancer, in particular malignant melanoma, and cancer of the skin, subcutaneous tissue, lymph nodes, brain, lung, liver, bone, intestine, colon, heart, pancreas, adrenals, kidney, prostate and breast, and to methods of reducing or avoiding adverse effects associated with anti-cancer agents such as temozolomide using thalidomide as adjunctive therapy. The invention also relates to pharmaceutical compositions and kits comprising temozolomide and thalidomide for use in combination therapy.

### 2. BACKGROUND OF THE INVENTION

The incidence of cancer continues to climb as the general population ages, as new cancers develop, and as susceptible populations (e.g., people infected with AIDS or excessively exposed to sunlight) grow. A tremendous demand therefore exists for new methods and compositions that can be used to treat patients with cancer.

### 2.1. Pathobiology of Cancer

Cancer is characterized primarily by an increase in the number of abnormal cells derived from a given normal tissue, invasion of adjacent tissues by these abnormal cells, or lymphatic or blood-borne spread of malignant cells to regional lymph nodes and to distant sites (metastasis). Clinical data and molecular biologic studies indicate that cancer is a multistep process that begins with minor preneoplastic changes, which may under certain conditions progress to neoplasia.

Pre-malignant abnormal cell growth is exemplified by hyperplasia, metaplasia, or most particularly, dysplasia (for review of such abnormal growth conditions, see Robbins and Angell, Basic Pathology, pp. 68-79, 2 d ed., W. B. Saunders Co., Philadelphia, 1976). Hyperplasia is a form of controlled cell proliferation involving an increase in cell number in a tissue or organ, without significant alteration in structure or function. As but one example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder.

The neoplastic lesion may evolve clonally and develop an increasing capacity for invasion, growth, metastasis, and heterogeneity, especially under conditions in which the neoplastic cells escape the host's immune surveillance. Roitt, I., Brostoff, J and Kale, D., Immunology, 17.1-17.12 (3rd ed., Mosby, St. Louis, Mo., 1993).

Descriptions of only a few types of cancers are provided below. Characteristics of other types of cancers are well known to medical practitioners, and are described in the medical literature.

### 2.2. Primary and Metastatic CNS Tumors

The incidence of primary and metastatic brain tumors is also increasing in the United States. Unfortunately, the arsenal of chemotherapeutics for these types of cancers is minimal, while the need for such therapeutics is high.

Glioblastoma multiforme and other primary and metastatic central nervous system tumors are devastating malignancies. The treatment of these tumors includes surgery, radiation therapy and treatment with agents such as the nitrosourea BCNU. Other chemotherapeutic agents utilized include procarbazine, vincristine, hydroxyurea and cisplatin. But even when all three modalities (surgery, radiation therapy and chemotherapy) are utilized, the average survival of patients with central nervous system malignancies is only about 57 weeks. Another example is the survival of patients with multiple brain metastases due to malignant melanoma. The median survival of patient with more than three brain lesions is less than six months. Clearly, new treatment approaches are needed both for patients with newly diagnosed primary and metastatic central nervous system tumors, as well as for patients with such tumors which are refractory to the above modalities.

### 2.3. Breast, Lung, Bladder and Prostate Cancers

In the United States, the cumulative risk of developing breast cancer is reportedly about 10.2 percent. The Merck Manual 1815 (16^{th} ed. 1992). The treatment for early breast cancer is surgery, with or without radiation therapy, or surgery, with or without radiation therapy, plus chemotherapy and/or hormonal therapy. Current chemotherapy for patients with primary or metastatic breast cancer includes treatment with cyclophosphamide, methotrexate, doxorubicin, 5-fluorouracil, cisplatin, vinblastine, taxol, taxotere, mitomycin C and occasionally other agents. Unfortunately, even with these agents, almost all women who develop metastatic breast cancer succumb to their disease. One particular place that metastatic breast cancer does metastasize to is the central nervous system. When central nervous system metastases do occur, the usual treatment is surgery (for a solitary metastasis) or radiation, or surgery plus radiation therapy.

Lung cancer is reportedly the leading cause of cancer death in men and women. The Merck Manual 731 (16^{th} ed. 1992). A variety of causes exist, but cigarette smoking accounts for greater than 90 percent of reported cases in men and greater than 70 percent of reported cases in women. Id.

Most patients with lung cancer present a tumor that has already metastasized to a variety of organs, including lung, liver, adrenal gland and other organs. Treatment of metastatic lung cancer is not yet standardized. Ihde, D.C., The New England Journal of Medicine 327:1434-1441 (1992). However, chemotherapy regimens that are utilized include treatment with cisplatin plus etoposide, combinations of cyclophosphamide plus doxorubicin plus cisplatin, and single agents alone or in combination, including ifosfamide, teniposide, vindesine, carboplatin, vincristine, taxol, nitrogen mustard, methotrexate, hexamethylmelamine and others. Despite these chemotherapeutic regimens the average patient with metastatic lung cancer still only survives 7-12 months. One particular troublesome place for metastases of lung cancer is the central nervous system. The treatment for central nervous system metastases includes surgery (to remove a solitary lesion), radiation therapy, or a combination of both.

Each year about 50,000 new cases of bladder cancer are reported in the United States. The Merck Manual 1749 (16^{th} ed. 1992). Although at presentation the disease is usually localized, most patients develop distant metastatic disease. The most recent advances have been in the area of chemotherapy for patients with such metastatic disease. One effective regimen is called the MVAC regimen. It consists of treatment with methotrexate plus vinblastine plus adriamycin (doxorubicin) plus cisplatin. Although the response rate is high to this chemotherapeutic regimen, medical oncologists are noting that one place the patients fail is with metastases to the central nervous system.

It is estimated that more than 120,000 men will be diagnosed with prostate cancer this year. The Merck Manual 1750 (16^{th} ed. 1992). The most common sites of metastases in patients with prostate cancer are the bone and lymph nodes. The bone metastases are particularly bothersome in that they can create intense pain for the patient. The current treatment for metastatic prostate cancer includes treatment with flutamide, leuprolide, diethylstilbestrol, and other hormonal manipulations, as well as chemotherapy (doxorubicin, estramustine phosphate, vinblastine, suramin, cisplatin, and others). Unfortunately, none of these agents are consistently helpful in the disease. In addition, as patients with prostate cancer live longer with their malignancy, they will most likely develop a higher incidence of metastases to the central nervous system (including the spinal cord).

### 2.4. Esophageal Cancer

Several years ago, carcinoma of the esophagus reportedly represented only about six percent of all cancers of the gastrointestinal tract; however, it reportedly caused a disproportionate number of cancer deaths. Boring, C. C., et al., CA Cancer J. Clin. 43:7 (1993). These cancers usually arise from the epithelial layer of the esophagus and are either squamous cell carcinomas or adenocarcinomas. Overall, the 5 year survival is about five percent.

### 2.5. Colorectal Cancer

In 1999, the incidence of colorectal cancer in the United States was 129,400 cases. In Western countries, the colon and rectum account for more new cases of cancer per year than any other anatomic site except the lung. The Merck Manual p. 852 (16^{th} ed., Mark H. Beers, M.D. and Robert Berkow, M.D. eds., Merck Research Laboratories, 1992). In the United States, about 75,000 people died of these cancers in 1989; about 70% occurred in the rectum and sigmoid, and 95% were adenocarcinomas. Colorectal cancer is the most frequent cause of death among visceral malignancies that affect both sexes. Id. The incidence begins to rise age 40 and peaks at age 60 to 75 yr. Cancer of the colon is more common in women; cancer of the rectum is more common in men. Synchronous cancers (more than one) occur in 5% of patients. Id. Most colorectal cancers are adenocarcinomas.

There is a low genetic predisposition to cancer of the large bowel, but “cancer families” and “colon cancer families” (e.g., familial polyposis, Lynch syndrome) are described, in which colorectal cancer occurs across several generations, usually present before age 40, and occurs more commonly in the right colon. As least four genes located on chromosomes 2, 3, and 7 have been shown to be mutated in some cases of the Lynch syndrome. Other predisposing factors include chronic ulcerative colitis, granulomatous colitis, and familial polyposis (which includes Gardner's syndrome); in these disorders, the risk of cancer at any time is related to the age of onset and duration of the underlying disease. Id.

Populations with a high incidence of colorectal cancer eat low-fiber diets that are high in animal protein, fat, and refined carbohydrates. Carcinogens may be ingested in the diet but are more likely to be produced from dietary substances or from biliary or intestinal secretions, probably by bacterial action. The exact mechanism is unknown. Colorectal cancer spreads by direct extension through the bowel wall, hematogenous metastasis, regional lymph node metastasis, perineural spread, and intraluminal metastasis. Id.

Primary treatment of colorectal cancers typically includes surgery. Many patients, however, must also be treated with a combination of radiation and chemotherapy. When surgery is not curative, limited palliative surgery may be indicated; median survival is 7 mo. As of 1999, the only drug with proven efficacy for advanced colorectal cancer using a chemotherapy regime was 5-fluorouracil (5FU), but only 15 to 20% of 5-FU patients experience demonstrable tumor shrinkage and prolongation of life. Other drugs, alone or with 5-FU, generally have not demonstrated better results. Id.

### 2.6. Skin Cancer

Skin cancer is the most common form of cancer, but most types of skin cancers are curable. The Merck Manual of Medical Information: Home Edition, p. 992 (Robert Berkow, M.D. ed., Merck Research Laboratories 1997). The more common forms of skin cancer usually develop on sun-exposed areas. Id. People who have had a lot of sun exposure, particularly people with fair complexions, are most likely to develop skin cancer. Id.

Melanoma is a cancer that originates in the pigment producing cells of the skin (melanocytes). About 25,000 new cases of malignant melanoma occur yearly in the United States, causing about 6,000 deaths. The Merck Manual, p. 843. The incidence is rising rapidly. Sun exposure is a risk, as is family history and the occurrence of lentigo maligna, large congenital melanocytic nevus, and the dysplastic nevus syndrome. Id. Melanoma can begin as a new, small, pigmented skin growth on normal skin, most often on sun-exposed areas, but nearly half of the cases develop from existing pigmented moles. The Merck Manual Of Medical Information, p. 993. The very rare malignant melanomas of childhood almost always arise from large pigmented moles present at birth. Signs of malignant transformation should be carefully sought: change in size, change in color, especially spread of red, white, and blue pigmentation to surrounding normal skin; change in surface characteristics, consistency, or shape and especially signs of inflammation in surrounding skin, with possible bleeding, ulceration, itching, or pain. The Merck Manual, p. 844. Unlike other forms of skin cancer, melanoma readily spreads (metastasizes) to distant parts of the body, where it continues to grow and destroy tissue. The Merck Manual of Medical Information, pp. 993-994.

The survival of patients with metastatic melanoma varies widely, ranging from only a few months to more than 10 years. Survival is primarily dependent on the sites of the first metastases, the number of metastatic sites, and responsiveness to treatment. Balch et al., “A Multifactorial Analysis of Melanoma,” J. Clin. Oncol., 1, 126 (1983). Melanoma can metastasize to virtually any organ or tissue. The initial sites of metastases, however, are most commonly the skin, soft tissue, lymph nodes, and lung. The liver, bone, and brain are also common, through less frequent sites of initial relapse. Patients with nonvisceral disease (i.e., skin, lymph nodes, and lung metastases) have a better median survival, ranging from 12 to 15 months, and are more likely to respond to systemic therapy. Patients with visceral disease (i.e., liver, bone, and brain metastases) have a median survival of only 3 to 4 months, and few respond to treatment. In general, cure is not a realistic goal of treatment at this stage of the disease. Therefore, treatment strategies must endeavor to preserve quality of life while attempting to prolong life. Id.

The less a melanoma has grown into the skin, the greater the chance of curing it. The clinical type of tumor is less important to the survival rate than the thickness of the tumor at the time of diagnosis. The Merck Manual of Medical Information, pp. 993-994. If a melanoma has grown deep into the skin, it's more likely to spread through the lymph and blood vessels and can cause death within months or a few years. Id. Local metastasis results in formation of nearby satellite papules or nodules that may or may not be pigmented. The Merck Manual, p. 845. Direct metastasis to skin or internal organs may occur, and occasionally metastatic nodules or enlarged lymph nodes are discovered before the primary lesion is identified. Id. Melanomas arising from mucous membranes have a poor prognosis, although they often seem quite limited when discovered. Id. The course of the disease varies greatly and appears to depend on the strength of the body's immune defenses. The Merck Manual of Medical Information, p. 994. Some people survive in apparent good health for many years despite the spread of the melanoma. Id.

When melanoma is suspected, a biopsy is performed. Small growths are removed entirely, but on a small piece is removed from larger growths. In either case, a pathologist examines the tissue microscopically to determine if the growth is a melanoma. Id.

Surgery can remove the entire melanoma; if the melanoma hasn't spread, the cure rate approaches 100 percent. However, anyone who has had a melanoma is at risk of developing others. Therefore, such people need regular skin examinations. Although chemotherapy is used to treat melanomas that have spread, cure rates are low, and the condition is often fatal. Id.

### 2.7. Temozolomide

The imidazotetrazines novel structure and chemistry as well as their encouraging antitumor activity in animal model systems has created much interest. Stevens, et al., “Antitumour activity and pharmacokinetics in mice of 8-carbamoyl-3-methyl-imidazo-[5.1.d]-1,2,3,5-tetrazin-4(3H)-one, a novel drug with potential as an alternative to dacarbazine,” 47 Cancer Res., 5846 (1987). Temozolomide, an imidazotetrazine second-generation alkylating agent, is the leading compound in a new class of chemotherapeutic agents that enter the cerebrospinal fluid and do not require hepatic metabolism for activation. Friedman et al., “Temozolomide and Treatment of Malignant Glioma,” 6 Clinical Cancer Research, 2585-2597 (2000). In preclinical studies, temozolomide demonstrated distribution to all tissues, including penetration into the central nervous system; relatively low toxicity compared with the parent compound, mitoxolomide; and antitumor activity against a broad range of tumor types. Id. Temozolomide is of particular interest because at physiological pH temozolomide undergoes chemical degradation to 5-(3-methyltriazen-1-yl)imidazole-4-carboxamide (MTIC) without the activation with subsequent formation of a reactive methyl-diazonium species. Hvizdos et al., “Temozolomide,” 12 CNS Drugs, 237-243 (1999).

Although clear inhibition of EMT6 mouse mammary-tumor esterases activity has been observed for temozolomide, it was less reactive than other agents such as 1,3-bis(2-chloroethyl)-2-nitrosourea (BCNU). Similar inhibition in intact cells and sonicated preparations indicated the cellular influx of the drug does not appear to be the limiting factor. Moreover, the inhibitory effect of temozolomide against esterases are weak compared with that of one or more potent carbamoylating agents such as BCNU. Dive, et al., 25 Cancer Chemother. Pharmacol., 149-155 (1989).

Once administered, temozolomide concentrations decline rapidly. After intravenous administration, plasma temozolomide concentrations declined biexponentially and having a half life distribution of 1.8 h. Newlands, et al., “Phase I trail of temozolomide (CCRG 81045; M&B 39831; NSC 362856),” 65 Br. J. Cancer, 287-291 (1992). Symptomatic toxicity from temozolomide on single dose schedule was mainly nausea and vomiting. This was mild at dosages up to 700 mg/m², but at higher doses patients experienced Grade 4 toxicity. Other deleterious side effects associated with the administration of temozolomide include alopecia; haematological toxicity, such as dose leukopenia, lymphopenia, limiting neutropenia, thrombocytopenia, and anemia; cardiovascular peripheral edema; central nervous system disorders such as amnesia, insomnia, paresthesia, somnolence, ataxia, dysphasia, convulsions, and confusion; gastrointestinal disorders such as abdominal pain, anorexia, constipation, diarrhea, gastrointestinal bleeding, liver enzyme abnormalities, nausea, stomatitis, vomiting; metabolic disorders such as asthenia, fatigue, fever, headache, and lethargy; respiratory disorders such as pharyngitis, pneumonia, and sinusitis; dermatological disorders such as skin rashes and mild erythematous; genitourinary disorders such as urinary track infections, increased urinary frequency, and incontinence; and ophthalmic disorders such as diplopia or visual disturbances; depletion of O⁶-guanine-alkyl-transferase; infection; raised transaminases; raised alkaline phosphatase; and raised bilirubin, with some side effects being severe in cachectic patients. Solimando et al., “Epirubin, Temozolomide,” 35 Hospital Pharmacy, 359-361 (2000). Although temozolomide has demonstrated antitumor activity against glioma, malignant glioma, melanoma, mesothelioma, sarcoma, lymphoma, leukemia, and carcinoma of the colon and ovary, effective methods for administering larger doses of temozolomide have yet to be successful. Additionally, to date no studies have been able to increase the effectiveness of temozolomide without increasing toxicity side-effects.

Temozolomide has been used in combination with 1,3-bis(2-chloroethyl)-1-nitrosourea (BCNU) to deplete cells or tumors of O⁶-alkylguanine-DNA alkyltransferase (AGT) and colon cancer. Mitchell et al., “Effect of temozolomide and dacarbazine on O⁶-alkylguanine-DNA alkyltransferase activity and sensitivity of human tumor cells and xenografts to 1,3-bis(2-chloroethyl)-1-nitrosourea,” 32 Cancer Chemother. Pharmacol., 59-63 (1993). Lower dosages of temozolomide did not enhance BCNU's depletion of AGT in colon tumor cells, higher dosages were required to achieve depletion. At these higher dosages, the combination of BCNU and temozolomide provided some synergistic effect, however, the data presented demonstrated that toxic doses of temozolomide were required to achieve significant AGT depletion. Id.

Administration of temozolomide for the treatment of refractory anaplastic astrocytoma has recently been approved in the United States. Temozolomide is administered at a dose of about 150 mg/m²/d and is an oral alternative to dacarbazine.

### 2.8. Thalidomide

Thalidomide is a racemic compound sold under the tradename THALOMID® and chemically named α-(N-phthalimido)glutarimide or 2-(2,6-dioxo-3 -piperidinyl)-1H-isoindole-1,3(2H)-dione. Thalidomide was originally developed in the 1950's to treat morning sickness, but due to its tetragenic effects was withdrawn from use. Thalidomide is now indicated in the United States for the acute treatment of the cutaneous manifestations of erythema nodosum leprosum. Physicians' Desk Reference, 911-916 (54^{th} ed., 2000). Because its administration to pregnant women can cause birth defects, the sale of thalidomide is strictly controlled. Id.

In addition to treating symptoms of leprosy, thalidomide has reportedly been used on patients with chronic graft-vs-host disease, rheumatoid arthritis, sarcoidosis, several inflammatory skin diseases, and inflammatory bowel disease. See generally, Koch, H. P., 22 Prog. Med. Chem. 165-242 (1985). See also, Moller, D. R., et al., 159 J. Immunol. 5157-5161 (1997); Vasiliauskas, E. A., et al., 117 Gastroenterology 1278-1287 (1999); and Ehrenpreis, E. D., et al., 117 Gastroenterology 1271-1277 (1999). It has further been alleged that thalidomide can be combined with other drugs to treat ischemia/reperfusion associated with coronary and cerebral occlusion. See U.S. Pat. No. 5,643,915, which is incorporated herein by reference.

Thalidomide is being clinically investigated in the treatment of specific types of cancers. These include refractory multiple myeloma, brain, melanoma, breast, colon, mesothelioma, and renal cell carcinoma. See, e.g., Singhal, S., et al., 341(21) New England J. Med, 1565-1571 (1999); and Marx, G. M., et al., 18 Proc. Am. Soc. Clin. Oncology, 454a (1999). It has further been reported that thalidomide can be used to prevent the development of chronic cardiomyopathy in rats caused by doxorubicin. Costa, P. T., et al., 92(10:suppl. 1) Blood, 235b (1998). Other reports concerning the use of thalidomide in the treatment of specific cancers include its combination with carboplatin in the treatment of glioblastoma multiforme. McCann, J., Drug Topics 41-42 (Jun. 21, 1999). Thalidomide has reportedly also been used as an antiemetic during the treatment of astrocytoma. Zwart, D., 16(12) Arzneim.-Forsch., 1688-1689(1966).

If there is a general mechanism by which thalidomide aids in the treatment of some cancers, its nature remains unclear. See, e.g., Moreira, A. L., et al., 177 J. Expr. Med 1675-1680 (1993); McHugh, S. M., et al., 99 Clin. Exper. Immunol., 160-167 (1995); and Moller, D. R., et al., 159 J. Immunol, 5157-5161 (1997). It has been reported, however, that thalidomide is an antiangiogenic agent that can suppress tumor necrosis factor a (TNF-α) and interleukin 12 (IL-12) production. See, e.g., Moller, D. R., et al., 159 J. Immunol., 5157-5161 (1997); Moreira, A. L., et al., 177 J. Exp. Med., 1675-1680 (1993); U.S. Pat. Nos. 5,593,990, 5,629,327, and 5,712,291 to D'Amato and U.S. Pat. No. 5,385,901 to Kaplan. And in vitro studies suggest that thalidomide affects the production of a variety of other proteins. See, e.g., McHugh, S. M., et al., 99 Clin. Exp. Immunol. 160-167 (1995). Thalidomide may also affect mechanisms related to epithelial or endothelial function or growth. D'Amato M., et al., 91 Proc. Natl. Acad. Sci. 4082-4085 (1994).

Given the great need for an effective and safe treatment of cancer, there continues to be an extensive amount of research on new drugs or ways of improving existing therapies. Efforts to improve management of malignant melanoma should focus on the development of new antitumor agents and novel combination regimens. Desirable characteristics of new treatment strategies include improved response in visceral metastases, penetration of the blood-brain barrier with activity in brain metastases, improved survival, and preservation of quality of life in the form of reduced toxicity, improved tolerability, and ease of administration. This invention addresses the need for a safe and effective cancer treatment, in particular for primary and metastatic malignant melanoma, and cancer of the skin, subcutaneous tissue, lymph nodes, brain, lung, liver, bone, intestine, colon, and related cancers.

### 3. SUMMARY OF THE INVENTION

This invention is directed to pharmaceutical compositions, pharmaceutical dosage forms and kits for the treatment of cancer using combination therapies. Further, the invention relates to methods of treating, preventing, or managing primary and/or metastatic cancer, in particular malignant melanoma, and cancer of the skin, subcutaneous tissue, ocular melanoma, lymph nodes, brain, lung, liver, bone, intestine, colon, heart, pancreas, adrenals, kidney, prostate, breast or combinations thereof, methods of reducing or avoiding adverse effects associated with certain chemotherapy and radiation therapy, and methods of improving the tolerance of patients to chemotherapy and radiation treatment for cancer.

A first embodiment of the invention encompasses a method of treating, preventing, or managing primary and/or metastatic cancer comprising administering to a patient in need of such treatment a therapeutically effective amount of temozolomide, or a pharmaceutically acceptable prodrug, salt, solvate, hydrate, or clathrate thereof, and a therapeutically effective amount of thalidomide, or a pharmaceutically acceptable prodrug, salt, solvate, hydrate, or clathrate thereof, administered prior, during, or after administering temozolomide.

Examples of cancers that can be treated by this method include, but are not limited to, cancer of the head, neck, eye, skin, mouth, throat, esophagus, chest, bone, lung, colon, sigmoid, rectum, stomach, prostate, breast, ovaries, kidney, liver, pancreas, brain, intestine, heart, adrenals, and combinations thereof. Specific cancers that can be treated by this method are malignant melanoma, and cancer of the skin, subcutaneous tissue, lymph nodes, brain, lung, liver, bone, intestine, colon, heart, pancreas, adrenals, kidney, prostate, breast or combinations thereof. A preferred method of this embodiment further comprises administering a maintenance dose of thalidomide, such as 50 to 200 mg/d, to a patient. In another preferred method of this embodiment, temozolomide is administered in an amount of from about 25 to about 500 mg/m²/d, preferably from about 50 to about 250 mg/m²/d, more preferably from about 50 to about 200 mg/m²/d, and even more preferably from about 100 to 200 mg/m²/d, and thalidomide is administered in an amount of from about 50 to 1000 mg/d, preferably from about 50 to 750 mg/d, and more preferably from about 50 to about 400 mg/d.

When administered to elderly patients (patients 65 to 70 years of age or older) temozolomide is administered in an amount of from about 25 to about 500 mg/m²/d, preferably from about 50 to about 250 mg/m²/d, and more preferably from about 50 to 200 mg/m²/d, and thalidomide is administered in an amount of from about 50 to 750 mg/d, preferably from about 50 to 500 mg/d, and more preferably from about 50 to about 250 mg/d.

A second embodiment of the invention encompasses a method of increasing the dosage of temozolomide, or a pharmaceutically acceptable prodrug, salt, solvate, hydrate, or clathrate thereof, that can be safely and effectively administered to a patient, which comprises administering to a patient in need of such an increased dosage an amount of thalidomide, or a pharmaceutically acceptable prodrug, salt, solvate, hydrate, or clathrate thereof, that is sufficient to reduce or avoid a dose-limiting adverse effect associated with temozolomide. Optionally, thalidomide is administered prior, during, or after administering temozolomide. In a preferred method of this embodiment, thalidomide is administered orally and daily in an amount of from about 1 to about 2000 mg, preferably from about 50 to about 1000 mg, more preferably from about 50 to 750 mg, and most preferably from about 50 to about 400 mg on a daily basis.

Examples of dose-limiting adverse effects associated with temozolomide include, but are not limited to: alopecia; haematological toxicity, such as dose leukopenia, lymphopenia, limiting neutropenia, thrombocytopenia, and anemia; cardiovascular toxicity; neurological toxicity such as amnesia, insomnia, paresthesia, somnolence, ataxia, dysphasia, convulsions, and confusion; gastrointestinal toxicity such as abdominal pain, anorexia, constipation, diarrhea, gastrointestinal bleeding, liver enzyme abnormalities, nausea, stomatitis, loss of appetite, low blood sugar, high blood sugar, vomiting; metabolic toxicity such as asthenia, fatigue, fever, headache, dizziness and lethargy; pulmonary toxicity such as pharyngitis, pneumonia, and sinusitis; dermatological toxicity such as skin rashes and mild erythematous; genitourinary toxicity such as urinary track infections, increased urinary frequency, and incontinence; and ophthalmic toxicity such as diplopia or visual disturbances; depletion of O⁶-guanine-alkyl-transferase; infection; raised transaminases; raised alkaline phosphatase; and raised bilirubin.

A third embodiment of the invention encompasses a method of reducing or preventing an adverse effect associated with cancer chemotherapy or radiation therapy, which comprises administering to a patient in need of such treatment or prevention an amount of temozolomide, or a pharmaceutically acceptable prodrug, salt, solvate, hydrate, or clathrate thereof, and thalidomide, or a pharmaceutically acceptable prodrug, salt, solvate, hydrate, or clathrate thereof, that is sufficient to reduce an adverse effect associated with the chemotherapy or radiation therapy. This embodiment includes the use of thalidomide to protect against or treat an adverse effect associated with the use of cancer chemotherapy or radiation therapy. Specific cancers that can be treated by this method are malignant melanoma, cancer of the skin, subcutaneous tissue, lymph nodes, brain, lung, liver, bone, intestine, colon, heart, pancreas, adrenals, kidney, prostate, breast, colorectal, or combinations thereof. The use of the thalidomide in this embodiment encompasses raising a patient's tolerance for chemotherapy or radiation therapy. In a preferred method of this embodiment, temozolomide is administered parenterally or orally in an amount of from about 25 to about 500 mg/m², preferably from about 50 to about 250 mg/m², and more preferably from about 50 to 200 mg/m² and thalidomide is administered orally in an amount of from about 1 to about 2000 mg, preferably from about 50 to about 1000 mg, more preferably from about 50 to 750 mg, and most preferably from about 50 to about 400 mg.

Examples of adverse effects associated with cancer chemotherapy and radiation therapy include, but are not limited to: anemia; anorexia; constipation; depletion of O⁶-guanine-alkyl-transferase; diarrhea; fatigue; gastrointestinal bleeding; headache; infection; lethargy; leukopenia; liver enzyme abnormalities; lymphopenia; nausea; neutropenia; pneumonia; raised transaminases; raised alkaline phosphatase; raised bilirubin; skin rash; stomatitis; urinary tract infection; thrombocytopenia; and vomiting.

A fourth embodiment of the invention encompasses a method of increasing the therapeutic efficacy of temozolomide, or a pharmaceutically acceptable prodrug, salt, solvate, hydrate, or clathrate thereof, which comprises administering to a patient in need thereof an amount of thalidomide, or a pharmaceutically acceptable prodrug, salt, solvate, hydrate, or clathrate thereof. Thalidomide is administered prior, during, or after administering temozolomide.

A fifth embodiment of the invention encompasses a pharmaceutical composition comprising temozolomide, or a pharmaceutically acceptable prodrug, salt, solvate, hydrate, or clathrate thereof, and thalidomide, or a pharmaceutically acceptable prodrug, salt, solvate, hydrate, or clathrate thereof. In a preferred composition of this embodiment, temozolomide is present in an amount of from about 25 to about 500 mg/m², preferably from about 50 to about 250 mg/m², and more preferably from about 50 to 200 mg/m² and thalidomide is present in an amount of from about 1 to about 2000 mg, preferably from about 50 to about 1000 mg, more preferably from about 50 to 750 mg, and most preferably from about 50 to about 400 mg.

A sixth embodiment of the invention encompasses a kit for use in the treatment or prevention of cancer which comprises a dosage form of temozolomide, or a pharmaceutically acceptable prodrug, salt, solvate, hydrate, or clathrate thereof, a dosage form of thalidomide, or a pharmaceutically acceptable prodrug, salt, solvate, hydrate, or clathrate thereof, and instructions for the use of each actor in combination for the treatment of prevention of cancer.

A seventh embodiment of the invention encompasses a method of reducing or preventing an adverse effect associated with the administration of thalidomide, which comprises administering to a patient in need of such treatment or prevention an amount of thalidomide, or a pharmaceutically acceptable prodrug, salt, solvate, hydrate, or clathrate thereof, and temozolomide, or a pharmaceutically acceptable prodrug, salt, solvate, hydrate, or clathrate thereof. Non-limiting examples of such adverse effects are birth defects, drowsiness, peripheral neuropathy, dermatological disorder, constipation, dry mouth, dry skin, swelling of the face or limbs, increased appetite, nausea, nervousness, ear buzzing or addiction to thalidomide.

### 3.1. Definitions

As used herein, the term “cancer” includes but is not limited to solid tumors and blood born tumors. The term “cancer” refers to disease of skin tissues, organs, blood, and vessels. The invention encompasses the treatment of various types of cancer including but not limited to cancer of the head, neck, eye, mouth, throat, esophagus, chest, bone, lung, colon, sigmoid, rectum, stomach, prostate, breast, ovaries, kidney, liver, pancreas, and brain. In a preferred embodiment, the invention encompasses the treatment of various types of cancer, including but not limited to malignant melanoma, and cancer of the skin, eye, subcutaneous tissue, lymph nodes, brain, lung, liver, bone, intestine, colon, heart, pancreas, adrenals, kidney, prostate, breast or combinations thereof, and more preferably, malignant melanoma, and cancer of the skin, subcutaneous tissue, lymph nodes, brain, or combinations thereof. In particular, the term “colorectal cancer” refers to disease of skin tissues, organs, bloods, and vessels, of the colon, sigmoid, and/or rectum and within the vicinity of the colon, sigmoid, and/or rectum. The term “cancer” further encompasses primary and metastatic cancers, unless otherwise indicated.

As used herein, unless otherwise specified, the term “preventing” includes but is not limited to, inhibition or the averting of symptoms associated with cancer. As used herein, unless otherwise specified, the term “treating” refers to the administration of a composition after the onset of symptoms of the cancer whereas “preventing” refers to the administration prior to the onset of symptoms, particularly to patients at risk of cancer, e.g., prevention of metastasis before they occur in patients with primary cancer.

As used herein to describe a compound or chemical moiety, the term “derivative” means a compound or chemical moiety wherein the degree of saturation of at least one bond has been changed (e.g., a single bond has been changed to a double or triple bond) or wherein at least one hydrogen atom is replaced with a different atom or a chemical moiety. Examples of different atoms and chemical moieties include, but are not limited to, halogen, oxygen, nitrogen, sulfur, hydroxy, methoxy, alkyl, amine, amide, ketone, and aldehyde.

As used herein, the term “prodrug” means a derivative of a compound that can hydrolyze, oxidize, or otherwise react under biological conditions (in vitro or in vivo) to provide the compound. Examples of prodrugs include, but are not limited to, derivatives of temozolomide or thalidomide that comprise biohydrolyzable moieties such as biohydrolyzable amides, biohydrolyzable esters, biohydrolyzable carbamates, biohydrolyzable carbonates, and biohydrolyzable ureides.

As used herein, the terms “optically pure,” “pure enantiomer,” and “optically pure enantiomer” mean a composition that comprises one enantiomer of a compound and is substantially free of another enantiomer of the compound. A typical optically pure enantiomeric composition comprises greater than about 80% by weight of one enantiomer of a compound and less than about 20% by weight of the other enantiomer of the compound, more preferably greater than about 90% by weight of one enantiomer of a compound and less than about 10% by weight of the other enantiomer of the compound, even more preferably greater than about 95% by weight of one enantiomer of a compound and less than about 5% by weight of the other enantiomer of the compound, and most preferably greater than about 99% by weight of one enantiomer of a compound and less than about 1% by weight of the other enantiomer of the compound.

### 4. DETAILED DESCRIPTION OF THE INVENTION

This invention encompasses pharmaceutical compositions, pharmaceutical dosage forms and kits for treating or preventing cancer with temozolomide and thalidomide. Further, the invention relates to methods of treating, preventing or managing diseases or conditions such as primary and/or metastatic cancer, methods of preventing metastases, methods of improving the therapeutic profile of either or both temozolomide or thalidomide, and methods of reducing or avoiding adverse effects associated with certain chemotherapy and radiation therapy.

This invention is based, in part, on the ability of a combination of temozolomide and thalidomide to: (1) treat cancer; (2) improve the efficacy of either drug when used alone against cancer or to improve the tolerability of either drug or perhaps other chemotherapeutic or radiation therapies for cancer; or (3) lessen the severity of certain dose-limiting toxicities of certain anti-cancer drugs. Without being limited by theory, it is believed that thalidomide exhibits antiangiogenic activity and other biological modulatory effects that may provide additive or synergistic antitumor effects when given concurrently with temozolomide. In particular, thalidomide has the potential to enhance the therapeutic efficacy of temozolomide when both are administered on an extended continuous schedule. The mechanism of the antitumor action of thalidomide may be related to antiangiogenesis. Because antiangiogenic drugs target the endothelial cells and not the tumor cells, the drugs could potentially be synergistic against chemotherapy-resistant tumors when combined with other cytotoxic agents. Thalidomide has a broad spectrum of activity, is available in well-tolerated oral form, has predictable side effects that are managed easily, and does not enhance the toxicity of other anti-cancer drugs.

Embodiments of the invention include a method of treating or preventing cancer, in particular malignant melanoma, cancer of the skin, subcutaneous tissue, eye, mucosal, lymph nodes, brain, lung, liver, bone, intestine, colon, heart, pancreas, adrenals, kidney, prostate, breast, colorectal, or combinations thereof, more preferably malignant melanoma, cancer of the skin, subcutaneous tissue, lymph nodes, brain, or combinations thereof. The method comprises the administration of temozolomide or a derivative, analogue, pharmaceutically acceptable prodrug, salt, solvate, hydrate, or clathrate thereof in combination with thalidomide, or a derivative, analogue, pharmaceutically acceptable prodrug, salt, solvate, hydrate, or clathrate thereof, either simultaneously or sequentially to a patient. Another embodiment of the invention encompasses a method of reducing or avoiding adverse effects associated with anti-cancer drugs, which comprises administering temozolomide, or a derivative, analogue, pharmaceutically acceptable prodrug, salt, solvate, hydrate, or clathrate thereof, and thalidomide, or a derivative, analogue, pharmaceutically acceptable prodrug, salt, solvate, hydrate, or clathrate thereof either simultaneously or sequentially to a patient.

Examples of other anti-cancer drugs that can be used in methods of the invention include, but are not limited to, Taxol® (paclitaxel), taxotere (docetaxel), doxorubicin, cisplatin, topoisomerase inhibitors, and other drugs described herein (e.g., those described below in Section 4.1.1.). Other embodiments of the invention encompass pharmaceutical compositions, pharmaceutical dosage forms, and kits comprising temozolomide, or a derivative, analogue, pharmaceutically acceptable prodrug, salt, solvate, hydrate, or clathrate thereof, and thalidomide, or a derivative, analogue, pharmaceutically acceptable prodrug, salt, solvate, hydrate, or clathrate thereof, and at least one other anti-cancer drug.

This invention further encompasses methods of: 1) allowing the completion of chemotherapy in a greater percentage of patients; 2) avoiding thrombocytopenia of patients' blood; and 3) improving the overall quality of patients' life during chemotherapy.

Preferred embodiments of the invention are based on the unique ability of thalidomide to improve the overall therapeutic profile of temozolomide when used in the treatment or prevention of various primary or metastatic cancers. For example, thalidomide as used in this invention can improve the efficacy of temozolomide at its common or approved doses. Thalidomide can further be used in combination with lower doses of temozolomide to reduce or avoid adverse affects associated with the administration of temozolomide while maintaining efficacy. Thalidomide can also be used in methods of this invention to reduce or avoid adverse effects that are associated with temozolomide. Indeed, a preferred use of thalidomide is to reduce or avoid intolerance of temozolomide so that temozolomide can be used in a greater amount in the treatment of cancer, in particular malignant melanoma, and cancer of the skin, subcutaneous tissue, lymph nodes, brain, or combinations thereof. And a specific embodiment of the invention encompasses the use of thalidomide to reduce or avoid thrombocytopenia toxicity caused by temozolomide. In yet another specific embodiment of the invention, comprises administering thalidomide in a maintenance dose after the combined administration of thalidomide and temozolomide. In short, this invention encompasses therapeutic effects that result from an unexpected and unique synergy between thalidomide and temozolomide. One of these therapeutic effects is an increased potency or efficacy of temozolomide; another is a reduced toxicity or increased safety of temozolomide.

Compositions of the invention include bulk drug compositions (e.g., impure or non-sterile compositions) useful in the manufacture of pharmaceutical compositions, pharmaceutical compositions (i.e., compositions that are suitable for administration to a patient), and individual dosage forms. Each of the compositions and dosage forms of the invention comprise at least two of what are referred to herein as “active ingredients.” A first active ingredient is temozolomide, a derivative or analogue of temozolomide, or a pharmaceutically acceptable prodrug, salt, solvate, hydrate, or clathrate thereof. A second active ingredient is thalidomide, a derivative or analogue of thalidomide, or a pharmaceutically acceptable prodrug, salt, solvate, hydrate, or clathrate thereof.

The synthesis of temozolomide is disclosed in U.S. Pat. No. 5,260,291, to Lunt et al. Temozolomide is available from CRC Experimental Chemotherapy Research Group, Aston University, Birmingham, UK; Drug Synthesis and Chemistry Branch, Division of Cancer Treatment, National Cancer Institute, Bethesda, Md.; and May and Baker Limited, Dagenham, Essex. Examples of derivatives and analogues of temozolomide that can be used in the methods and compositions of the invention include, but are not limited to, the compounds disclosed in U.S. Pat. No. 5,260,291, which are incorporated herein by reference. It is further contemplated that pharmaceutically acceptable prodrugs, salts, solvates, clathrates, and derivatives of temozolomide be used in the methods and compositions of the invention.

Thalidomide contains a chiral center, and is sold as a racemate. The methods and compositions of the invention therefore encompass the use of racemic thalidomide as well as optically pure enantiomers of thalidomide. Optically pure enantiomers of thalidomide can be prepared by methods well known in the art. These include, but are not limited to, resolution of chiral salts, asymmetric synthesis, or chiral chromatography. It is further contemplated that pharmaceutically acceptable prodrugs, salts, solvate, clathrates and derivatives of thalidomide be used in the methods and compositions of the invention. Examples of derivatives of thalidomide that can be used in the methods and compositions of the invention include, but are not limited to, taglutimide, supidimide, EM-12, and those disclosed by International Application WO 94/20085, which is incorporated herein by reference. Other derivatives of thalidomide encompassed by this invention include, but are not limited to, 6-alkyl-2-[3′- or 4′-nitrophthalimido]-glutarimides and 6-alkyl-3-phenylglutarimides. See, e.g., De, A. U., and Pal. D., 64(2) J. Pharm. Sci., 262-266 (1975). Preferred thalidomide derivatives are the amino analogues of thalidomide such as amino-thalidomide and the compounds disclosed in U.S. Pat. No. 5,463,063, which are incorporated herein by reference.

Another embodiment of the invention is a method of reducing or preventing an adverse effect associated with the administration of thalidomide, which comprises administering to a patient in need of such treatment or prevention an amount of thalidomide, or a pharmaceutically acceptable prodrug, salt, solvate, hydrate, or clathrate thereof, and temozolomide, or a pharmaceutically acceptable prodrug, salt, solvate, hydrate, or clathrate thereof. Non-limiting examples of such adverse effects are birth defects, drowsiness, peripheral neuropathy, dermatological disorder, constipation, dry mouth, dry skin, swelling of the face or limbs, increased appetite, nausea, nervousness, ear buzzing or addiction to thalidomide.

### 4.1. Methods of Treatment and Prevention

This invention encompasses methods of treating and preventing a variety of disease and conditions in mammals, and in humans in particular. Although dosage forms of the invention can be used in methods of the invention, the active ingredients disclosed herein can be administered simultaneously or sequentially, i.e., separately, in any appropriate form, and by any suitable route.

Without being limited by theory, it is believed that the combined use of temozolomide and thalidomide to a patient suffering from cancer provides a unique and unexpected synergism. In particular, and without being limited by theory, it is believed that thalidomide exhibits antiangiogenic activity and other biological modulatory effects that may provide additive or synergistic antitumor effects when given concurrently with chemotherapy. Thalidomide can work in combination with temozolomide to more rapidly kill cancer cells, while at the same time reducing thrombocytopenia and other side effects associated with chemotherapy and radiation therapy.

Consequently, one embodiment of this invention encompasses methods of treating and/or preventing of cancer. Examples of cancers that can be treated are disclosed herein and include, but are not limited to, primary and metastatic cancer of the head, neck, eye, skin, mouth, throat, esophagus, chest, bone, lung, stomach, prostate, breast, ovaries, kidney, liver, pancreas, brain, intestine, colon, heart, adrenals, rectum, sigmoid, and surrounding tissues. Specific examples of cancers that can be treated include, but are not limited to: malignant melanoma, cancer of the skin, subcutaneous tissue, lymph nodes, brain, lung, liver, bone, intestine, colon, heart, pancreas, adrenals, kidney, prostate, breast, colorectal, or combinations thereof. More specific examples of cancers that can be treated include, malignant melanoma, cancer of the skin, subcutaneous tissue, lymph nodes, brain, and combinations thereof.

The invention encompasses methods of treating of patients with primary and metastatic cancers. It further encompasses methods of treating patients who have been previously treated for cancer, as well as those who have not previously been treated for cancer. Indeed, the methods and compositions of this invention can be used in first-line and second-line cancer treatments. In a specific embodiment of the invention, the cancer is metastatic. In another specific embodiment, the patient having cancer is immunosuppressed by reason of having previously undergone anti-cancer therapy (e.g., chemotherapy radiation). In a preferred embodiment, thalidomide is administered to a patient undergoing temozolomide treatment before any adverse effect or intolerance occurs.

Other embodiments of the invention include methods of increasing the dosage of temozolomide that can be safely and effectively administered to a patient, and methods of varying the dosage cycle used to administer temozolomide to a patient while avoiding dose-limiting toxicities.

Another embodiment of the invention described in detail encompasses a method of reducing, treating and/or preventing adverse, or undesired, effects associated with chemotherapy and/or radiation therapy.

### 4.1.1. Methods of Treating and/or Preventing Cancer

The methods of treating and/or preventing cancer encompassed by this invention comprise administering at least two drugs. Alternatively, the methods of treating and/or preventing cancer may include chemotherapy, radiation therapy, at least one additional anti-cancer drug, or a combination thereof.

One method of treating and/or preventing cancer comprises administering at least two drugs (also referred to herein as “active ingredients” or “active agents”) to a patient (e.g., a human) suffering, or likely to suffer, from cancer: 1) temozolomide, a derivative or analogue of temozolomide, or a pharmaceutically acceptable salt, solvate, clathrate, hydrate, or prodrug thereof and; 2) thalidomide, a derivative or analogue of thalidomide, or a pharmaceutically acceptable salt, solvate, clathrate, hydrate, or prodrug thereof. The two active ingredients can be administered concurrently or sequentially, and by the same or by different routes of administration. For example, one active ingredient (e.g., thalidomide) can be administered to a patient prior to, during, or after the administration of the other active ingredient (e.g. temozolomide) or vice versa.

Another method of treating and/or preventing cancer further comprises administering a maintenance dose of thalidomide after combination treatment to a patient (e.g., a human) suffering, or likely to suffer from malignant melanoma, cancer of the skin, subcutaneous tissue, lymph nodes, brain, or combinations thereof: 1) temozolomide, or a pharmaceutically acceptable derivative, prodrug, salt, solvate, hydrate, or clathrate thereof; and 2) thalidomide, or a pharmaceutically acceptable derivative, prodrug, salt, solvate, hydrate, or clathrate thereof wherein the two active ingredients are administered either concurrently or sequentially, and by the same or by different routes of administration. Preferably, both drugs are administered by the oral route.

Another method of treating and/or preventing cancer comprises administering at least two drugs to a patient suffering, or likely to suffer, from cancer and treating the patient with either chemotherapy or radiation therapy. The patient may be treated with chemotherapy or radiation therapy prior, during, or after drug administration.

A preferred method of treating and/or preventing cancer comprises administering at least two drugs to a patient (e.g., a human) suffering, or likely to suffer, from malignant melanoma, cancer of the skin, subcutaneous tissue, lymph nodes, brain, lung, liver, bone, intestine, colon, heart, pancreas, adrenals, kidney, prostate, breast, colorectal, or combinations thereof: 1) temozolomide, or a pharmaceutically acceptable derivative, prodrug, salt, solvate, hydrate, or clathrate thereof; and 2) thalidomide, or a pharmaceutically acceptable derivative, prodrug, salt, solvate, hydrate, or clathrate thereof wherein the two active ingredients are administered either concurrently or sequentially, and by the same or by different routes of administration.

Another embodiment of the invention encompasses a method of treating cancer which comprises the administration of at least three active ingredients simultaneously or sequentially: 1) temozolomide, or a pharmaceutically acceptable derivative, prodrug, salt, solvate, hydrate, or clathrate thereof; 2) thalidomide, or a pharmaceutically acceptable derivative, prodrug, salt, solvate, hydrate, or clathrate thereof; and 3) an additional anti-cancer drug. The active ingredient administration order can be concurrently or sequentially, wherein no particular order is followed in the sequence.

In yet another preferred embodiment of the invention, the method of treating and/or preventing cancer further comprises administering a maintenance dose of thalidomide, such as 50 to 200 mg/d.

Examples of anti-cancer drugs that can be used in the various embodiments of the invention, including pharmaceutical compositions and dosage forms and kits of the invention, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O⁶-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin. These two agents are particularly useful when used in methods employing temozolomide and thalidomide.

The magnitude of a prophylactic or therapeutic dose of each active ingredient in the acute or chronic management of cancer will typically vary with the severity of the cancer and the route of administration. The dose, and perhaps the dose frequency, may also vary according to age, body weight, response, and the past medical history of the patient. Suitable dosing regimens can be readily selected by those skilled in the art with due consideration of such factors by following, for example, dosages reported in the literature and recommended in the Physician's Desk Reference® (54^{th} ed., 2000; 55^{th} ed., 2001 or 56^{th} ed., 2002).

Unless otherwise indicated, the magnitude of a prophylactic or therapeutic dose of each active ingredient used in an embodiment of the invention will be that which is known to those in the art to be safe and effective, or is regulatory approved.

In one embodiment of the invention, temozolomide is administered orally or parenterally during about five days in a four or six week cycle in an amount of from about 1 to about 750 mg/m²/day, preferably in an amount of from about 25 to about 500 mg/m²/day, more preferably in an amount of from about 50 to about 250 mg/m²/day, and most preferably in an amount of from about 50 to about 200 mg/m²/day. In another embodiment, temozolomide is administered at different dosages such as about two to about six dose levels, preferably about four dose levels, for 6 weeks followed by a rest period. The rest period can be from about two to about six weeks, preferably from about four, three, or two weeks. Another embodiment of the invention, thalidomide is administered orally and daily in an amount of from about 1 to about 2000 mg, preferably from about 50 to about 1000 mg, more preferably from about 50 to 750 mg, and most preferably from about 50 to about 400 mg. In another embodiment, thalidomide is administered daily for two weeks, followed by an increase in dose at two week intervals until a maximum daily dose of about 400 mg is reached.

As noted elsewhere herein, this invention encompasses a method of reducing the time between therapeutically safe and effective doses of anti-cancer drugs. Consequently, in one specific embodiment of the invention, temozolomide is administered in a five day schedule with a four week cycle followed by a break of about three weeks (e.g., about once every three weeks, about once every two weeks, or about once every ten days). The invention further allows the frequency, number, and length of anti-cancer drug dosing cycles to be increased. Thus, another specific embodiment of the invention encompasses the administration of temozolomide for more cycles than are typical when it is administered alone. See, e.g., Dhodapkar, et. al., “Phase I Trial of Temozolomide (NSC 362856) in Patients with Advanced Cancer,” 3 Clinical Cancer Research, 1093-1100 (1997). In yet another specific embodiment of the invention, temozolomide is administered for a greater number of cycles that would typically cause dose-limiting toxicity in a patient to whom thalidomide is not also being administered.

In one embodiment, temozolomide and thalidomide are administered in combination during a cycle of 8-10 weeks. In this cycle, temozolomide is administered daily and continuously for 6 weeks at a dose of 50 to 75 mg/m²/d followed by a break of 1, 2, 3 or 4 weeks. Thalidomide is administered daily and continuously throughout the 8-10 week cycle at an initial dose of 200 mg/d with dose escalation (every 2 weeks) by 100 mg/d to a maximum dose of 400 mg/d for as long as therapy is tolerated. Elderly patients (patients 65 to 70 years of age or older) are administered thalidomide at an initial dose of 100 mg/d with dose escalation (every 2 weeks) by 50 mg/d to a maximum dose of 200 mg/d for as long as therapy is tolerated.

In one embodiment of the invention, temozolomide and thalidomide are administered orally, with administration of thalidomide occurring 30 to 60 minutes prior to temozolomide.

In another embodiment of the invention, the combination of temozolomide and thalidomide is administered by intravenous infusion over about 90 minutes every cycle. In a specific embodiment one cycle comprises the administration of about 400 mg/m²/day (250 mg of temozolomide and 150 mg of thalidomide) daily for five days and then three weeks of rest. In another specific embodiment, each cycle comprises the administration of about 400 mg/m²/day (200 mg of temozolomide and 200 mg of thalidomide) followed by three sk weeks of rest. Typically, the number of cycles during which the combinatorial treatment is administered to a patient will be from about 1 to about 12 cycles, more typically from about 2 to about 10 cycles, and even more typically from about 2 to about 8 cycles.

The dosage amounts and frequencies provided above are encompassed by the terms “therapeutically effective,” “prophylactically effective,” and “therapeutically or prophylactically effective” as used herein. When used in connection with an amount of thalidomide or thalidomide derivative, these terms further encompass an amount of thalidomide or thalidomide derivative that reduces, prevents, or eliminates an adverse effect associated with the administration of radiation or an anti-cancer drug such as temozolomide, or an amount that otherwise improves the efficacy of radiation therapy or of an anti-cancer drug in the treatment or prevention of cancer, in particular colorectal cancer.

The suitability of a particular route of administration employed for a particular active ingredient will depend on the active ingredient itself (e.g., whether it can be administered orally without decomposing prior to entering the blood stream) and the disease being treated. For example, treatment of tumors on the skin or on exposed mucosal tissue may be more effective if one or both active ingredients are administered topically, transdermally, or mucosally (e.g., by rectal administration). Treatment of tumors within the body, or prevention of cancer that may spread from one part of the body to another, may be more effective if one or both of the active ingredients are administered parenterally or orally. Similarly, parenteral administration may be preferred for the acute treatment of cancer, whereas transdermal or subcutaneous routes of administration may be employed for chronic treatment or prevention of cancer.

It should be noted that the combination of thalidomide and temozolomide can be used in both male and female patients, in the elderly (e.g., patients over 65) and in children and adults. Further, the invention including the use of combination in patients who have previously received other chemotherapy or radiation treatment for cancer as well as patients not previously treated. In a preferred embodiment, the combination is used in patients with brain metastases from melanoma, including patients previously treated with dacarbazine, tamoxifen, cisplatin, carmustine or combinations thereof. Preferably, these chemotherapeutics are stopped while the combination of temozolomide and thalidomide are used.

It is preferred that both thalidomide and temozolomide are used orally. Further, thalidomide is preferably started at 100 mg/d for patients over 65 or 70 and 200 mg/d for patients under 65-70. Thalidomide is then titrated at 50 mg increments per week for the elderly and 100 mg increments per week for others until 250 mg/d or 400 mg/d are reached, respectively.

### 4.1.2. Methods of Increasing Anti-cancer Drug Dosages

This invention encompasses a method of increasing the dosage of an anti-cancer drug, such as temozolomide, that can be safely and effectively administered to a patient. This method comprises administering to a patient (e.g., a human) thalidomide, or a pharmaceutically acceptable derivative, salt, solvate, clathrate, hydrate, or prodrug thereof. Patients that can benefit by this method are those likely to suffer from an adverse effect associated with drugs for treating malignant melanoma, cancer of the skin, subcutaneous tissue, lymph nodes, brain, lung, liver, bone, intestine, colon, heart, pancreas, adrenals, kidney, prostate, breast, colorectal, or combinations thereof, e.g., temozolomide, that is alleviated or reduced by the administration of thalidomide, or a pharmaceutically acceptable derivative, salt, solvate, clathrate, hydrate, or prodrug thereof, and which is of such severity that it would otherwise limit the amount of temozolomide that can be safely and effectively administered to them. Such adverse effects are referred to herein as “dose-limiting.”

For example, adverse effects that are associated with temozolomide and which can limit the amount of temozolomide that can safely and effectively be administered to a patient include, but are not limited to: alopecia; haematological toxicity, such as dose leukopenia, lymphopenia, limiting neutropenia, thrombocytopenia, and anemia; cardiovascular peripheral edema; central nervous system disorders such as amnesia, insomnia, paresthesia, somnolence, ataxia, dysphasia, convulsions, and confusion; gastrointestinal disorders such as abdominal pain, anorexia, constipation, diarrhea, gastrointestinal bleeding, liver enzyme abnormalities, nausea, stomatitis, vomiting; metabolic disorders such as asthenia, fatigue, fever, headache, and lethargy; respiratory disorders such as pharyngitis, pneumonia, and sinusitis; dermatological disorders such as skin rashes and mild erythematous; genitourinary disorders such as urinary track infections, increased urinary frequency, and incontinence; and ophthalmic disorders such as diplopia or visual disturbances; depletion of O⁶-guanine-alkyl-transferase; infection; raised transaminases; raised alkaline phosphatase; and raised bilirubin.

According to a specific method of the invention, thalidomide, or a pharmaceutically acceptable derivative, salt, solvate, clathrate, hydrate, or prodrug thereof, is administered prior to, during, or after temozolomide, or a pharmaceutically acceptable derivative, salt, solvate, clathrate, hydrate, or prodrug thereof. In one embodiment, thalidomide is administered orally and daily in an amount of from about 1 to about 2000 mg, preferably from about 50 to about 1000 mg, more preferably from about 50 to 750 mg, and most preferably from about 50 to about 400 mg.

### 4.1.3. Methods of Treating and/or Preventing Adverse Effects Associated with Chemotherapy and Radiation Therapy

As discussed elsewhere herein, this invention encompasses a method of treating and/or preventing adverse effects associated with chemotherapy and/or radiation therapy, such as that administered to patients with malignant melanoma, cancer of the skin, subcutaneous tissue, lymph nodes, brain, lung, liver, bone, intestine, colon, heart, pancreas, adrenals, kidney, prostate, breast, colorectal, or combinations thereof. This method comprises administering to a patient (e.g., a human) thalidomide, or a pharmaceutically acceptable derivative, salt, solvate, clathrate, hydrate, or prodrug thereof before, during, or after the occurrence of the adverse effect.

Examples of adverse effects associated with chemotherapy and radiation therapy that can be treated or prevented by this method include, but are not limited to: anemia; anorexia; constipation; depletion of O⁶-guanine-alkyl-transferase; diarrhea; fatigue; gastrointestinal bleeding; headache; infection; lethargy; leukopenia; liver enzyme abnormalities; lymphopenia; nausea; neutropenia; pneumonia; raised transaminases; raised alkaline phosphatase; raised bilirubin; skin rash; stomatitis; urinary tract infection; thrombocytopenia; and vomiting.

According to this method, thalidomide, or a pharmaceutically acceptable derivative, salt, solvate, clathrate, hydrate, or prodrug thereof, is administered prior to, during, or after chemotherapy or radiation therapy. In one embodiment of this method, thalidomide is administered prior to the administration of temozolomide or radiation therapy. In another embodiment, thalidomide is administered during or after the administration of temozolomide or radiation therapy. In still another embodiment, thalidomide is administered at least twice for each treatment with temozolomide or radiation therapy; e.g., once during the treatment and at least once following the treatment, once prior to the treatment and once during the treatment, once both prior to and at least once after the treatment, or combinations thereof. Preferably, thalidomide is administered before any adverse event or symptom occurs. Indeed, thalidomide can be administered to a patient prior to the administration of chemotherapy and/or radiation therapy, in which case it can be considered as a protectant.

In a specific embodiment of this method, thalidomide is administered in an amount of from about 1 to about 2000 mg, preferably from about 50 to about 1000 mg, more preferably from about 50 to 750 mg, and most preferably from about 50 to about 400 mg orally and daily following radiation therapy or the administration of temozolomide.

### 4.2. Pharmaceutical Compositions and Dosage Forms

Pharmaceutical compositions can be used in the preparation of individual dosage forms. Consequently, pharmaceutical compositions and dosage forms of the invention comprise the active ingredients disclosed herein (i.e., temozolomide, or a pharmaceutically acceptable prodrug, salt, solvate, hydrate, or clathrate thereof; and thalidomide, a derivative or analogue of thalidomide, or a pharmaceutically acceptable prodrug, salt, solvate, hydrate, or clathrate thereof). Pharmaceutical compositions and dosage forms of the invention can further comprise one or more excipients.

Pharmaceutical compositions and dosage forms of the invention can also comprise one or more additional active ingredients. Examples of optional additional active ingredients include those enumerated in section 4.1.1 above.

Single unit dosage forms of the invention are suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of malignant melanoma, cancer of the skin, subcutaneous tissue, lymph nodes, brain, lung, liver, bone, intestine, colon, heart, pancreas, adrenals, kidney, prostate, breast, colorectal, or combinations thereof may contain larger amounts of one or more of the active ingredients it comprises than a dosage form used in the chronic treatment of cancer. Similarly, a parenteral dosage form may contain smaller amounts of one or more of the active ingredients it comprises than an oral dosage form used to treat malignant melanoma, cancer of the skin, subcutaneous tissue, lymph nodes, brain, lung, liver, bone, intestine, colon, heart, pancreas, adrenals, kidney, prostate, breast, colorectal, or combinations thereof. These and other ways in which specific dosage forms encompassed by this invention will vary from one another will be readily apparent to those skilled in the art. See, e.g., Remington's Pharmaceutical Sciences, (18th ed., Mack Publishing, Easton Pa., 1990).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, in embodiments comprising at least one additional active ingredient, other than temozolomide and thalidomide, the decomposition of the active ingredients may be accelerated by some excipients such as lactose, or when exposed to water. Active ingredients that comprise primary or secondary amines are particularly susceptible to such accelerated decomposition. Consequently, this invention encompasses pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions of the invention can comprise excipients that are well known in the art and are listed, for example, in the U.S. Pharmocopia (USP) SP (XXI)/NF (XVI). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 379-80 (2d. Ed., Marcel Dekker, NY, N.Y., 1995). In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms of the invention comprise thalidomide, a derivative or analogue of thalidomide, or a pharmaceutically acceptable salt, solvate, clathrate, hydrate, or prodrug thereof in an amount of from about 1 mg to about 2000 mg, more preferably from about 50 mg to about 1000 mg, even more preferably from about 50 mg to about 750 mg, and most preferably from about 50 mg to about 400 mg. Similarly, typical dosage forms of the invention comprise temozolomide or a pharmaceutically acceptable salt, solvate, clathrate, hydrate, prodrug or derivative thereof in an amount of from about 1 mg to about 750 mg, more preferably from about 25 mg to about 500 mg, even more preferably from about 50 mg to about 250 mg, and most preferably from about 50 mg to about 200 mg.

### 4.2.1. Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, (18th ed., Mack Publishing, Easton Pa. 1990).

Typical oral dosage forms of the invention are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W. R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

A preferred solid oral dosage form of the invention comprises temozolomide, thalidomide, anhydrous lactose, microcrystalline cellulose, polyvinylpyrrolidone, stearic acid, colloidal anhydrous silica, and gelatin.

### 4.2.2. Delayed Release Dosage Forms

Active ingredients of the invention can be administered by controlled release means or by delivery devices that are well known to those of ordinary skill in the art. Examples include, but are not limited to, those described in U.S. Pat. Nos.: 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

### 4.2.3. Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention. For example, cyclodextrin and its derivatives can be used to increase the solubility of thalidomide, temozolomide, and their derivatives. See, e.g., U.S. Pat. No. 5,134,127, which is incorporated herein by reference.

A preferred parenteral composition of the invention is intended for dilution with 5% Dextrose Injection, USP, or 0.9% Sodium Chloride Injection, USP, prior to administration to a patient, and is an aqueous solution that comprises temozolomide, thalidomide, sorbitol NF powder, and lactic acid, USP, and has a pH of from about 3.0 to about 3.8.

### 4.2.4. Transdermal, Topical, and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, (16th and 18th eds., Mack Publishing, Easton Pa. 1980 & 1990); and Introduction to Pharmaceutical Dosage Forms, (4th ed., Lea & Febiger, Philadelphia 1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, (16th and 18th eds., Mack Publishing, Easton Pa. 1980 & 1990).

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### 4.2.5. Kits

Typically, active ingredients of the invention are preferably not administered to a patient at the same time or by the same route of administration. This invention therefore encompasses kits which, when used by the medical practitioner, can simplify the administration of appropriate amounts of active ingredients to a patient.

A typical kit of the invention comprises a dosage form of an anti-cancer drug, i.e., temozolomide, or a pharmaceutically acceptable prodrug, salt, solvate, hydrate, or clathrate thereof, and a dosage form of thalidomide, or a pharmaceutically acceptable derivative, prodrug, salt, solvate, hydrate, or clathrate thereof. Kits encompassed by this invention can further comprise additional active ingredients. Examples of optional additional active ingredients include those mentioned in section 4.1.1.

Kits of the invention can further comprise devices that are used to administer the active ingredients. Examples of such devices include, but are not limited to, syringes, drip bags, patches, and inhalers.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Kits of the invention can further comprise tissue culture of the tumor cells and assay methods to determine the tumor cell's susceptibility to thalidomide, temozolomide, or both.

A specific kit of the invention comprises a solid dosage form of thalidomide suitable for oral administration to a patient, and a solution dosage form of temozolomide suitable for parenteral administration to a patient. A preferred oral dosage form of thalidomide comprises 50 mg thalidomide, anhydrous lactose, microcrystalline cellulose, polyvinylpyrrolidone, stearic acid, colloidal anhydrous silica, and gelatin.

Other kits encompassed by the invention will be readily apparent to those skilled in the art, since thalidomide, temozolomide, and other therapeutic and anti-cancer drugs or radiation therapies are well known and/or commercially available. More specifically, kits can also contain instructions for the use of thalidomide in combination with temozolomide such as dosing, cycling, monitoring, etc. in accordance with the description found herein.

### 5. EXAMPLES

Certain embodiments of the invention are illustrated by the following non-limiting examples.

### 5.1. EXAMPLE 1

### Treatment of Cancer

A 43-year-old woman with metastatic melanoma in the brain, lung, and subcutaneous soft tissue who had developed new leptomeningeal disease after whole-brain radiation for bilateral multiple lesions was treated. After receiving two cycles of the combination therapy, the patient had near complete response in all sites of metastases, resolution of multiple hemorrhagic intracranial lesions, diffuse leptomeningeal disease, resolution of subcutaneous nodules, and reduction in lung nodules. The patient received the temozolomide and thalidomide combination therapy for a period of one year at which point the disease was determined to be in complete remission.

### 5.2. EXAMPLE 2

### Treatment of Cancer

After completing one year of high-dose adjuvant interferon alfa therapy, a 39-year-old woman developed metastatic melanoma in the brain, lung, mediastinum, spleen, and pelvis. The patient was treated using the combination therapy of temozolomide and thalidomide. After one cycle, the patient exhibited response in the pituitary stalk and multiple intracranial lesions, near resolution of the large mediastinal mass, and significant reductions in splenic and pelvic disease.

### 5.3. EXAMPLE 3

### Treatment of Malignant Melanoma

One patient, a 60-year-old woman who developed multiple in-transit metastases in the leg and metastases in the inguinal lymph nodes 37 years after a primary melanoma was removed from her ankle, was treated initially with inguinal lymphadenectomy and isolated limb perfusion with caboplatin (paraplatin). Postoperatively, the patient received adjuvant high-dose interferon therapy, but her disease recurred in the leg shortly after completion of 1 year of adjuvant therapy; treatment included isolated limb perfusion with melphalan (Alkeran) and tumor necrosis factor. Subsequently, her disease progressed in the pelvis and retroperitoneum and interleukin-2 systemic therapy was used. However, disease progression continued not only in the leg, pelvis, and retroperitoneum, but new metastases also developed in the liver and mesentery with massive malignant ascites.

On initial presentation, the patient was cachectic with a distend abdomen and had a Karnofsky performance status of 40. She was started on thalidomide at 100 mg/d and dacarbazine on an every-3-week schedule. After 3 months, not only did the ascites completely resolve, but significant shrinkage of metastases in the liver and mesentery was observed, the retroperitoneal adenopathy had completely resolved and the pelvic adenopathy bad markedly improved.

The patient elected to discontinue dacarbazine, but remained on thalidomide at a maximum dose of 200 mg/d. After an additional 3 months, resolution of the metastases continued and virtually no adenopathy was detected. The single treatment with thalidomide continued for one year, with only recurrence in the leg.

### 5.4. EXAMPLE 4

### Treatment of Metastatic

### Melanoma in the Brain

A group consisting of 16 patients (median age, 57 years; range, 26-79) having brain metastases from melanoma were treated with a combination of temozolomide and thalidomide. Temozolomide was administered at 75 mg/m²/d continuously for 6 weeks, followed by a 2 week break, with continuous daily administration of thalidomide. Thalidomide was administered at an initial dose of 200 mg/d with a dose escalation of 100 mg/d at 2 week intervals to a maximum dose of 400 mg/day. Elderly patients (≧70 years) were administered thalidomide at an initial dose of 100 mg/d with a dose escalation of 50 mg/d at 2 week intervals to a maximum does of 250 mg/day. Treatment continued with all patients until toxicity or disease progression occurred. Response to treatment was evaluated after each 8-week cycle of therapy by brain CT of MRI scan. Radiographic response was defined as at least 25% reduction in tumor size, while disease progression was defined as te appearance of new tumors or an increase of more than 25% in tumor size.

Radiographic response was demonstrated in 6 of 16 patients with 3 patients achieving nearly complete resolution of all cerebral lesions. Stable disease was observed in 3 additional patients. The median follow-up time among survivors was 7.9 months and the median overall survival was 6.9 months. Additionally, 4 of the 6 patients with extracranial metastases showed radiographic evidence of clinical response. The combination treatment was well tolerated with no treatment-related grade 3 or 4 toxicities (grade 2 toxicities primarily associated with thalidomide included somnolence, headache, constipation, dry mouth, dry skin and rash).

### 5.5. EXAMPLE 5

### Treatment of Patients

### with Adavanced Melanoma

A pilot study was conducted to investigate the safety and efficacy of temozolomide using an extended, continuous schedule in combination with thalidomide. Thalidomide was administered at 200 mg/d for the first 2 weeks, followed by an increase of 100 mg/d at 2-week intervals until a maximum dose of 400 mg/d was reached. Temozolomide was administered at four dose levels consisting of 50 mg/m²/d for 6 weeks followed by a 4-week rest period, and 75 mg/m²/d for weeks followed by rest periods of 4, 3, or 2 weeks.

Eligible patients for this study were adults with biopsy-confirmed unresectable stage III or IV melanoma without brain metastases. A total of 12 patients were divided into 4 groups designated as Levels 1-4. Patients in Level 1 were administered temozolomide at a dose of 50 mg/m²/d for 6 weeks followed by a 4 week break. Patients in Levels 2, 3 and 4 were administered temozolomide at a dose of 75 mg/m²/d for 6 weeks followed, respectively, by breaks of 4, 3 or 2 weeks. Patients <70 years of age were administered thalidomide at an initial dose of 200 mg/day, and escalated in 100 mg increments at 2-week intervals to a maximum dose of 400 mg/day. Elderly patients (≧70 years) were administered thalidomide at an initial dose of 100 mg/day, and escalated in 50 mg increments at 2-week intervals to a maximum dose of 250 mg/day. Tumor response was evaluated every 8 weeks and treatment continued until disease progression or unacceptable toxicity.

Five major responses (1 complete, 4 partial) were documented, all at does levels 2-4. Three of the 5 responding patients were in the over 70 age group. The median duration of response was 6 months (range 4-12+months), the median overall survival was not reached (4-12+months), and the median follow-up among surviving patients was 12 months (9+-16+months).

The treatment regimen, summary of patient characteristics and outcome, response by dose level and summary of safety results for this example are shown below in Tables 1-4, respectively.

TABLE 1
Treatment Regimen

| DL | Study Treatment | Wk 1 | 2 | 3 | 4 | 5 | 6 | 7 | 8 | 9 | 10 |
|---|---|---|---|---|---|---|---|---|---|---|---|
| 1 | Temozolomide (mg/m²/d) | 50 | 50 | 50 | 50 | 50 | 50 | — | — | — | — |
| | Thalidomide* (mg/d; <70 y) | 200 | 200 | 300 | 300 | 400 | 400 | 400 | 400 | 400 | 400 |
| | (mg/d; ≧70 y) | 100 | 100 | 150 | 150 | 200 | 200 | 200 | 200 | 200 | 200 |
| 2 | Temozolomide (mg/m²/d) | 75 | 75 | 75 | 75 | 75 | 75 | — | — | — | — |
| | Thalidomide | | | | Same as dose level 1 | | | | | | |
| 3 | Temozolomide (mg/m²/d) | 75 | 75 | 75 | 75 | 75 | 75 | — | — | — | New |
| | Thalidomide | | | | Same as dose level 1 | | | | | | Cycle |
| 4 | Temozolomide (mg/m²/d) | 75 | 75 | 75 | 75 | 75 | 75 | — | — | New | |

| | | |
|---|---|---|
| Thalidomide | Same as dose level 1 | Cycle |

DL = dose level.
Wk = week.
*Thalidomide is escalated to a maximum dose of 400 mg/d (250 mg/d in patients ≧70 years) administered continuously as tolerated.

TABLE 2
Summary of Patient Characteristics and Outcome

| | Sex/ | | Prior | No. of | | OS | |
|---|---|---|---|---|---|---|---|
| DL | Age/PS | Number & Sites of Disease | Therapy | Cycles | OR | (mo) | Status |
| 1 | M/59/90 | 4 (LN, lung, liver) | S | 1 | MR | 6 | DOD |
| | M/75/80 | 5 (lung, liver, LN, ST) | None | 0 | NE | 4 | DOD |
| | M/47/90 | 2 (lung) | None | 1 | PD | 16+ | AWD |
| 2 | F/69/80 | 2 (lung, LN) | S, V | 3 | PR | 13+ | NED* |
| | M/73/90 | 1 (scalp) | None | 4 | CR | 12+ | NED |
| | M/75/90 | 7 (lung, in-transit, ST, LN) | S | 1 | Mix | 8 | DOD |
| 3 | F/47/90 | 4 (cutaneous, ST, LN) | S, V | 3 | PR | 12+ | AWD |
| | M/67/90 | 5 (lung, ST, LN) | S, V | 2 | PD | 11+ | AWD |
| | M/70/70 | 8 (lung, liver, spleen, | None | 2 | PR | 5 | DOD |
| | | adrenal, mesentary/ | | | | | |
| | | omentum, carcinomatosis, | | | | | |
| | | ST, malginant ascites) | | | | | |
| 4 | F/72/90 | 4 (lung, LN, liver, bone) | None | 3 | PR | 11+ | AWD |
| | M/61/90 | 5 (lung, liver, ST, LN, | V | 1 | PD | 5 | DOD |
| | | in-transit) | | | | | |
| | M/73/80 | 3 (lung, ST, LN) | S | 1 | SD | 9+ | AWD |

DL = dose level;
M = male;
F = female;
PS = Karnofsky performance status (%);
LN = lymph nodes;
ST = soft tissue;
S = surgery;
V = vaccine;
OR = objective response;
MR = minor response;
NE = not evaluable;
PD = progressive disease;
PR = partial response;
CR = complete response;
Mix = mixed response;
SD = stable disease;
OS = overall survival;
mo = months;
DOD = deceased of disease;
AWD = alive with disease;
NED = no evidence of disease.
*Patient underwent surgical resection of residual disease following temozolomide/thalidomide therapy.

TABLE 3
Response by Dose Level

| Dose Level | CR | PR | SD | PD |
|---|---|---|---|---|
| 1 | — | — | 1 | 1 |
| 2 | 1 | 1 | 1 | — |
| 3 | — | 2 | — | 1 |
| 4 | — | 1 | 1 | 1 |
| Total | 1 | 4 | 3 | 3 |

CR = complete response;
PR = partial response;
SD = stable disease;
PD = progressive disease

TABLE 4
Summary of Safety Results

| | No. of Patients |
|---|---|
| Toxicity | (n = 12) |
| Grade 2 leukopenia | 1 |
| Grade 3 DVT | 1 |
| Grade 4 PE | 1 |
| Grade 2/3 constipation | 8/1 |
| Grade 2 dyspnea | 3 |
| Grade 2/3 neuropathy | 2/2 |
| Grade 2/3 rash | 1/1 |
| Grade 2 nausea/vomiting | 1/1 |
| Grade 2 blurred vision | 1 |
| Grade 2 abdominal pain | 1 |
| or cramping | |
| Grade 2 dry skin | 1 |
| Grade 2 diarrhea | 1 |
| Grade 2 fatigue | 1 |
| Grade 2 headache | 1 |
| Grade 2 joint pain | 1 |
| Dose modification/ | 6/3 |
| discontinuation of | |
| thalidomide | |

DVT = deep vein thrombosis;
PE = pulmonary embolism

The embodiments of the invention described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

What is claimed is:

1. A method of treating primary cancer which comprises administering to a patient in need of such treatment a therapeutically effective amount of temozolomide, or a pharmaceutically acceptable prodrug, salt, solvate, hydrate, or clathrate thereof, and a therapeutically effective amount of thalidomide, or a pharmaceutically acceptable prodrug, salt, solvate, hydrate, or clathrate thereof.

2. A method of treating metastatic cancer which comprises administering to a patient in need of such treatment a therapeutically effective amount of temozolomide, or a pharmaceutically acceptable prodrug, salt, solvate, hydrate, or clathrate thereof, and a therapeutically effective amount of thalidomide, or a pharmaceutically acceptable prodrug, salt, solvate, hydrate, or clathrate thereof.

3. The method according to claim 1 or 2, wherein the cancer is primary malignant melanoma or metastatic melanoma.

4. The method according to claim 3, wherein the primary malignant melanoma is dermatological, ocular or mucosal.

5. The method according to claim 3, wherein the metastatic melanoma is of the skin, subcutaneous tissue, lymph nodes, lung, liver, spleen, adrenal gland, intestine, bone, brain, heart or kidney.

6. The method according to claim 3, further comprising administering a maintenance dose of thalidomide.

7. The method according to claim 6, wherein the maintenance dose of thalidomide is 50 to 200 mg/d.

8. The method according to claim 3, wherein the temozolomide, or pharmaceutically acceptable prodrug, salt, solvate, hydrate, or clathrate thereof, is administered in an amount of from about 50 to about 250 mg/m², and the thalidomide, or pharmaceutically acceptable prodrug, salt, solvate, hydrate, or clathrate thereof, is administered in an amount of from about 50 to about 1000 mg.

9. The method according to claim 8, wherein the temozolomide, or pharmaceutically acceptable prodrug, salt, solvate, hydrate, or clathrate thereof, is administered in an amount of from about 50 to about 200 mg/m², and the thalidomide, or pharmaceutically acceptable prodrug, salt, solvate, hydrate, or clathrate thereof, is administered in an amount of from about 50 to about 750 mg.

10. The method according to claim 9, wherein the temozolomide, or pharmaceutically acceptable prodrug, salt, solvate, hydrate, or clathrate thereof, is administered in an amount of from about 50 to about 200 mg/m², and the thalidomide, or pharmaceutically acceptable prodrug, salt, solvate, hydrate, or clathrate thereof, is administered in an amount of from about 50 to about 400 mg.

11. The method according to claim 1 or 2, wherein the temozolomide, or pharmaceutically acceptable prodrug, salt, solvate, hydrate, or clathrate thereof, is administered in an amount of about 75 mg/m²/d.

12. The method according to claim 1 or 2, wherein the thalidomide, or pharmaceutically acceptable prodrug, salt, solvate, hydrate, or clathrate thereof, is administered at an initial dose of from about 100 to about 200 mg/d.

13. The method according to claim 1 or 2, wherein the thalidomide, or pharmaceutically acceptable prodrug, salt, solvate, hydrate, or clathrate thereof, is administered to patient over the age of 70 at an initial dose of from about 50 to about 100 mg/d.

14. The method according to claim 1 or 2, wherein the thalidomide, or a pharmaceutically acceptable prodrug, salt, solvate, hydrate, or clathrate thereof, is administered prior to the administration of the temozolomide.

15. The method according to claim 1 or 2, wherein the thalidomide, or a pharmaceutically acceptable prodrug, salt, solvate, hydrate, or clathrate thereof, is administered simultaneously with the administration of the temozolomide.

16. The method according to claim 1 or 2, wherein the thalidomide, or a pharmaceutically acceptable prodrug, salt, solvate, hydrate, or clathrate thereof, is administered after the administration of the temozolomide.

17. The method according to claim 1 or 2, wherein the thalidomide is administered daily and continuously.

18. The method according to claim 1 or 2, wherein the temozolomide is administered daily and continuously for 6 weeks followed by a rest period.

19. The method according to claim 1 or 2, wherein the thalidomide, or pharmaceutically acceptable prodrug, salt, solvate, hydrate, or clathrate thereof, is administered in an amount of from about 50 to about 1000 mg.

20. The method according to claim 1 or 2, wherein the thalidomide, or pharmaceutically acceptable prodrug, salt, solvate, hydrate, or clathrate thereof, is administered in an amount of from about 50 to about 400 mg.

21. The method according to claim 1 or 2, wherein the thalidomide, or pharmaceutically acceptable prodrug, salt, solvate, hydrate, or clathrate thereof, is administered in an amount of from about 100 to about 200 mg.

22. A method of reducing or preventing an adverse effect associated with the administration of temozolomide, which comprises administering to a patient in need of such treatment or prevention an amount of temozolomide, or a pharmaceutically acceptable prodrug, salt, solvate, hydrate, or clathrate thereof, and thalidomide, or a pharmaceutically acceptable prodrug, salt, solvate, hydrate, or clathrate thereof.

23. The method according to claim 22, wherein the cancer is primary malignant melanoma or metastatic melanoma.

24. The method according to claim 23, wherein the primary malignant melanoma is dermatological, ocular or mucosal.

25. The method according to claim 23, wherein the metastatic melanoma is of the skin, subcutaneous tissue, lymph nodes, lung, liver, spleen, adrenal gland, intestine, bone, brain, heart or kidney.

26. The method according to claim 22, wherein the thalidomide, or a pharmaceutically acceptable prodrug, salt, solvate, hydrate, or clathrate thereof, is administered prior to the administration of the temozolomide.

27. The method according to claim 23, wherein the thalidomide, or a pharmaceutically acceptable prodrug, salt, solvate, hydrate, or clathrate thereof, is administered simultaneously with the administration of the temozolomide.

28. The method according to claim 22, wherein the thalidomide, or a pharmaceutically acceptable prodrug, salt, solvate, hydrate, or clathrate thereof, is administered after the administration of the temozolomide.

29. The method according to claim 22, wherein the adverse effect is alopecia, haematological toxicity, cardiovascular toxicity, neurological toxicity, gastrointestinal toxicityr, metabolic toxicity, pulmonary toxicity, dermatological toxicity, genitourinary toxicity or ophthalmic toxicity.

30. The method according to claim 29, wherein the adverse effect is leukopenia, lymphopenia, neutropenia, thrombocytopenia, anemia, paresthesia, somnolence, ataxia, dysphasia, confusion, abdominal pain, anorexia, constipation, diarrhea, liver enzyme abnormalities, nausea, vomiting, asthenia, fatigue, headache, lethargy, skin rashes, erythematous, visual disturbances, dizziness, nausea, headache, loss of apetite, low blood sugar, high blood sugar and infection.

31. The method according to claim 22, wherein the thalidomide, or pharmaceutically acceptable prodrug, salt, solvate, hydrate, or clathrate thereof, is administered in an amount of from about 50 to about 1000 mg.

32. The method according to claim 31, wherein the thalidomide, or pharmaceutically acceptable prodrug, salt, solvate, hydrate, or clathrate thereof, is administered in an amount of from about 50 to about 400 mg.

33. The method according to claim 32, wherein the thalidomide, or pharmaceutically acceptable prodrug, salt, solvate, hydrate, or clathrate thereof, is administered in an amount of from about 100 to about 200 mg.

34. A method of increasing the therapeutic efficacy of temozolomide which comprises administering to a patient in need thereof an amount of thalidomide, or a pharmaceutically acceptable prodrug, salt, solvate, hydrate, or clathrate thereof, that is sufficient to increase the therapeutic efficacy of the temozolomide.

35. The method according to claim 34, wherein the thalidomide, or a pharmaceutically acceptable prodrug, salt, solvate, hydrate, or clathrate thereof, is administered prior to administration of the temozolomide to the patient.

36. The method according to claim 34, wherein the thalidomide, or a pharmaceutically acceptable prodrug, salt, solvate, hydrate, or clathrate thereof, is administered during administration of the temozolomide to the patient.

37. The method according to claim 34, wherein the thalidomide, or a pharmaceutically acceptable prodrug, salt, solvate, hydrate, or clathrate thereof, is administered after administration of the temozolomide to the patient.

38. A kit for use in the treatment of cancer which comprises a dosage form of temozolomide or a pharmaceutically acceptable prodrug, salt, solvate, hydrate, or clathrate thereof, and a dosage form of thalidomide, or a pharmaceutically acceptable prodrug, salt, solvate, hydrate, or clathrate thereof.

39. The kit according to claim 38, further comprising a diagnostic assay to determine a patent's susceptibility to the treatment.

40. A pharmaceutical composition for treating or preventing primary or metastatic cancer comprising a therapeutically effective amount of temozolomide, or a pharmaceutically acceptable prodrug, salt, solvate, hydrate, or clathrate thereof, and a therapeutically effective amount of thalidomide, or a pharmaceutically acceptable prodrug, salt, solvate, hydrate, or clathrate thereof.

41. A method of increasing the dosage of temozolomide that can be safely and effectively administered to a patient, which comprises administering to a patient in need of such an increased dosage an amount of thalidomide, or a pharmaceutically acceptable prodrug, salt, solvate, hydrate, or clathrate thereof, that is sufficient to reduce a dose-limiting adverse effect associated with the temozolomide.

42. The method according to claim 41, wherein the dose-limiting adverse effect is selected from the group consisting of alopecia, haematological toxicity, cardiovascular toxicity, neurological toxicity, gastrointestinal toxicityr, metabolic toxicity, pulmonary toxicity, dermatological toxicity, genitourinary toxicity or ophthalmic toxicity.

43. A method of reducing or preventing an adverse effect associated with the administration of thalidomide, which comprises administering to a patient in need of such treatment or prevention an amount of thalidomide, or a pharmaceutically acceptable prodrug, salt, solvate, hydrate, or clathrate thereof, and temozolomide, or a pharmaceutically acceptable prodrug, salt, solvate, hydrate, or clathrate thereof.

44. The method of claim 43, wherein the adverse effect is birth defects, drowsiness, peripheral neuropathy or dermatological disorder.

45. The method of claim 43, wherein the adverse effect is constipation, dry mouth, dry skin, swelling of the face or limbs, increased appetite, nausea, nervousness, ear buzzing or addiction to thalidomide.
